(19) United States
(12) Patent Application Publication
(10) Pub. No.: US 2017/0145430 A1
(43) Pub. Date: May 25, 2017

(54) TRANSGENIC PLANT PRODUCTION METHOD FOR PRODUCING GENETICALLY MODIFIED PLANT AND GENETICALLY MODIFIED TRANSGENIC PLANT PRODUCED BY SAID PRODUCTION METHOD

(71) Applicant: SUMITOMO RUBBER INDUSTRIES, LTD., Kobe-shi (JP)

(72) Inventors: Chiharu TANI, Kobe-shi (JP); Akari OKADA, Kobe-shi (JP); Yukino INOUE, Kobe-shi (JP)

(73) Assignee: SUMITOMO RUBBER INDUSTRIES, LTD., Kobe-shi (JP)

(21) Appl. No.: 15/335,223

(22) Filed: Oct. 26, 2016

(30) Foreign Application Priority Data
Nov. 24, 2015 (JP) 2015-228872

(51) Int. Cl. C12N 15/82; A01H 4/00

(52) U.S. Cl. CPC C12N 15/8205; A01H 4/008; C12N 15/8209

(57) ABSTRACT

Provided is a transgenic plant production method for producing a genetically modified rubber-producing plant that is capable of efficient short-term gene transfer, of introducing a target gene while retaining the useful traits of a plant undergoing gene transfer, and of efficient redifferentiation. The method includes: a culture step of culturing a tissue fragment from a rubber-producing plant to obtain a cultured tissue fragment; a transformation step of transforming, with a gene construct, the cultured tissue fragment obtained in the culture step; and a regeneration step of regenerating a plant from the tissue fragment transformed in the transformation step, wherein the transformation step includes: an infection step of culturing the cultured tissue fragment in the presence of an Agrobacterium transformed with a gene construct; and a decontamination step of culturing the cultured tissue fragment obtained in the infection step in a decontamination medium containing a carbapenem antibiotic to decontaminate the Agrobacterium.

### TECHNICAL FIELD

The present invention relates to a transgenic plant production method for producing a genetically modified plant and further relates to a genetically modified transgenic plant produced by the production method.

### BACKGROUND ART

Much research has been carried out into the production of transgenic plants by the introduction of genes into plants, and successful examples have been reported for many plant species.

The breeding of woody plants for variety improvement is generally carried out by cross breeding, but woody plants require several years from the seed-grown seedling to flowering and fruiting, and depending on the case a long period of time, in units of several tens of years, is required. Genetic analysis thus requires longer periods of time than for herbaceous plants.

The genetic manipulation of plants, on the other hand, provides great potential for improving commercially important plant species. The genetic manipulation of trees has also been the subject of a variety of investigations in recent years and is used in particular in the pulp and timber industries.

At the present time, the natural rubber (a type of polyisoprenoid) used for industrial rubber products is obtained by the cultivation of rubber-producing plants, e.g. Para rubber tree (Hevea brasiliensis) of the family Euphorbiaceae or Indian rubber tree (Ficus elastica) among Moraceae plants; the laticifer cells in these plants biosynthesize natural rubber, and this natural rubber is collected from the plants by manual procedures.

Natural rubber for industrial applications is at present sourced almost entirely from Hevea brasiliensis. Moreover, it is used widely and in large amounts in a variety of applications as the main raw material for rubber products. However, Hevea brasiliensis is a plant that can be grown only in limited regions such as Southeast Asia and South America. Furthermore, Hevea brasiliensis requires about seven years from planting to becoming mature enough for rubber extraction, and the collection season is limited in some cases. The time period during which natural rubber can be collected from the mature tree is also limited to 20 to 30 years.

For the future, an increase in the demand for natural rubber, centering on developing countries, can be expected and the exhaustion of natural rubber resources is a concern, and thus a stable source of supply for natural rubber is desired.

Under these circumstances, efforts designed to increase the production of natural rubber by Hevea brasiliensis have appeared. With Hevea brasiliensis, seedling propagation is carried out by raising and growing seedlings provided by seeding to yield a rootstock and grafting buds obtained from clone plantlets to the rootstock. Since there are limitations on the buds obtained from clone plantlets, the mass propagation of improved clone plantlets is necessary in order to spread improved clones.

With regard to examples of investigations into the application of gene manipulation techniques, examples have been reported of the successful production of a transformant of Hevea brasiliensis using a somatic embryogenesis system in which a somatic embryo is induced after gene transfer of callus derived from a tissue such as an immature embryo, followed by redifferentiation to regenerate a plant (see, for example, Non-Patent Literature 1.

### CITATION LIST

### NON PATENT LITERATURE

Non-Patent Literature 1: R. Jayashree and 12 others, “Plant Cell Reports”, 2003, vol. 22, pp. 201-209

### SUMMARY OF INVENTION

### Technical Problem

As noted above, the production of a transformant of the woody plant Hevea brasiliensis using a somatic embryogenesis system has been carried out, but this method has a low transformation efficiency and is unstable and/or has a poor redifferentiation efficiency, and there has been room for improvement in terms of providing an effective transformation method that yields transgenic plants having a desired genotype.

Moreover, when an immature embryo from a fertilized seed is used, individuals having the same traits as the parent cannot be obtained, and as a consequence it has been difficult to maintain the quality of varieties.

Accordingly, for the difficult-to-redifferentiate woody plants, there is a desire for a gene transfer method that is capable of efficient short-term gene transfer and of introducing a target gene while retaining the useful traits of the plants, and there is also a desire for a transformant production method capable of efficient redifferentiation.

The present invention solves the aforementioned problems, and an object of the present invention is to provide a transgenic plant production method for producing a genetically modified rubber-producing plant that is capable of efficient short-term gene transfer, of introducing a target gene while retaining the useful traits of a plant undergoing gene transfer, and of efficient redifferentiation.

### Solution to Problem

Conventionally, gene transfer of woody plants has been carried out through adventitious shoot induction or somatic embryo induction from, e.g., the petiole, lamina, hypocotyl, and so forth. However, the production of transgenic plants by gene transfer has been difficult in some varieties for which redifferentiation of the individuals is difficult and formation of adventitious shoots or somatic embryos is difficult.

As a result of thoroughgoing investigations, the present inventors discovered that a genetically modified transgenic plant can be efficiently produced in a short period of time by carrying out transformation using an Agrobacterium and a cultured tissue fragment obtained by culturing a tissue fragment from a rubber-producing plant, and regenerating a plant from the transformed tissue fragment obtained by this transformation. Further, the present inventors believe that one important factor in increasing the efficiency of producing the genetically modified transgenic plant is to increase the efficiency of gene transfer into the cultured tissue fragment by Agrobacterium infection, and it is important to efficiently select tissue fragments that have undergone Agrobacterium infection and transformation. In this regard, in Agrobacterium-based gene transfer methods, the infection of the cultured tissue fragment with Agrobacterium is generally followed by the decontamination of the Agrobacterium using a medium containing a decontaminating agent that shows an antibacterial or bactericidal activity for Agrobacterium. However, the following considerations were discovered: if the Agrobacterium is not thoroughly decontaminated and a large amount of Agrobacterium is left, this can lead to a decline in the survival rate of the transformed tissue fragment; adverse effects can also appear, on the other hand, when the cultured tissue fragment is kept in contact with a high concentration of the decontaminating agent over a long period of time in order to thoroughly decontaminate the Agrobacterium. In view of these, it was concluded with regard to the decontamination of Agrobacterium that it is desirable to achieve a thorough decontamination in as short a period of time as possible and to use a low concentration of decontaminating agent. Then, as a result of thoroughgoing investigations, the present inventors discovered that, when a carbapenem antibiotic is used to decontaminate Agrobacterium, a low concentration of the antibiotic can thoroughly decontaminate the Agrobacterium in a short period of time. The present inventors came up with the idea that, by using this antibiotic to minimize the adverse effects on the plant arising from decontamination of the Agrobacterium, the decline in the survival rate of the transformed tissue fragment can be prevented and as a result the efficiency of producing the genetically modified transgenic plant can be increased, thereby completing the present invention.

Specifically, the present invention relates to a transgenic plant production method for producing a genetically modified plant, the production method including:
a culture step of culturing a tissue fragment derived from a rubber-producing plant to obtain a cultured tissue fragment;
a transformation step of transforming, with a gene construct, the cultured tissue fragment obtained in the culture step; and
a regeneration step of regenerating a plant from the tissue fragment transformed in the transformation step,
wherein the transformation step includes:
an infection step of culturing the cultured tissue fragment in the presence of an Agrobacterium that has been transformed with a gene construct; and
a decontamination step of culturing the cultured tissue fragment obtained in the infection step in a decontamination medium containing a carbapenem antibiotic to decontaminate the Agrobacterium.

The carbapenem antibiotic is preferably meropenem.

The carbapenem antibiotic is preferably present in the decontamination medium at a concentration of 10 to 500 mg/L.

Preferably, the gene construct contains a selective marker gene that confers resistance to a selective reagent, and the production method further includes a selective culture step of culturing the cultured tissue fragment decontaminated in the decontamination step in a selective culture medium containing the selective reagent to select the tissue fragment transformed in the transformation step.

The selective reagent is preferably paromomycin.

The selective reagent is preferably present in the selective culture medium at a concentration of 25 to 300 mg/L.

The cultured tissue fragment is preferably a shoot.

The rubber-producing plant is preferably a plant belonging to the genus Hevea.

Preferably, the production method further includes, after the culture step, a propagation step of collecting and subdividing the cultured tissue fragment obtained in the culture step, and culturing the subdivided cultured tissue fragment in an induction medium containing a plant growth hormone and a carbon source to obtain a cultured tissue fragment, and the cultured tissue fragment obtained in the propagation step is subjected to the transformation step.

The gene construct may contain genetic material that is homologous to the genome of the rubber-producing plant, or may contain genetic material that is heterologous to the genome of the rubber-producing plant.

The regeneration step preferably includes a rooting step of culturing the transformed tissue fragment in a rooting induction medium to induce rooting.

The present invention further relates to a genetically modified transgenic plant, produced by the above-described production method.

### Advantageous Effects of Invention

The transgenic plant production method for producing a genetically modified plant of the present invention includes: a culture step of culturing a tissue fragment derived from a rubber-producing plant to obtain a cultured tissue fragment; a transformation step of transforming, with a gene construct, the cultured tissue fragment obtained in the culture step; and a regeneration step of regenerating a plant from the tissue fragment transformed in the transformation step, wherein the transformation step includes: an infection step of culturing the cultured tissue fragment in the presence of an Agrobacterium that has been transformed with a gene construct; and a decontamination step of culturing the cultured tissue fragment obtained in the infection step in a decontamination medium containing a carbapenem antibiotic to decontaminate the Agrobacterium. Such a production method can efficiently produce a genetically modified transgenic plant in a short period of time.

### DESCRIPTION OF EMBODIMENTS

The production method of the present invention includes: a culture step of culturing a tissue fragment derived from a rubber-producing plant to obtain a cultured tissue fragment; a transformation step of transforming, with a gene construct, the cultured tissue fragment obtained in the culture step; and a regeneration step of regenerating a plant from the tissue fragment transformed in the transformation step, wherein the transformation step includes: an infection step of culturing the cultured tissue fragment in the presence of an Agrobacterium that has been transformed with a gene construct; and a decontamination step of culturing the cultured tissue fragment obtained in the infection step in a decontamination medium containing a carbapenem antibiotic to decontaminate the Agrobacterium. As described above, with such a transgenic plant production method for producing a genetically modified plant, a transgenic rubber-producing plant can be efficiently produced in a short period of time. This method is characterized particularly by using a carbapenem antibiotic to decontaminate the Agrobacterium after a cultured tissue fragment of a rubber-producing plant is transformed by an Agrobacterium-based gene transfer method. Due to this feature it is possible to reduce the concentration of the decontaminating agent used to decontaminate the Agrobacterium to low levels and to thoroughly decontaminate the Agrobacterium in a short period of time. It is therefore possible to minimize the adverse effects on the plant arising from decontamination of the Agrobacterium to prevent a decline in the survival rate of the transformed tissue fragment, thereby increasing the efficiency of producing the genetically modified transgenic plant. Thus, a genetically modified transgenic plant can be efficiently produced in a short period of time.

The production method of the present invention may include other steps as long as it includes the aforementioned steps. Each of the aforementioned steps may be performed once or may be carried out a plurality of times by, for example, subculture.

There are no particular limitations on the tissue fragment derived from a rubber-producing plant used in the culture step, and examples include petioles, laminas, hypocotyls of somatic embryos, nodes, axillary buds, apical buds, and so forth. Among these, the tissue fragment is preferably a tissue containing a node, axillary bud, or apical bud because then a cultured tissue fragment, preferably a shoot, can be stably induced. Thus, the culture step is preferably a step of culturing a rubber-producing plant-derived tissue containing a node, axillary bud, or apical bud to obtain a cultured tissue fragment, preferably a shoot.

There are no particular limitations on the rubber-producing plant, and examples include woody plants and herbaceous plants, from which rubber can be harvested. Specific examples include: the genus Hevea, e.g. Hevea brasiliensis; the genus Ficus, e.g. Ficus carica, Ficus elastica, Ficus pumila L., Ficus erecta Thumb., Ficus ampelas Burm. f., Ficus benguetensis Merr., Ficus irisana Elm., Ficus macrocarpa L. f., Ficus septica Burm. f., and Ficus benghalensis; Parhenium argentatum; and the genus Taraxacum, e.g. Taraxacum, Taraxacum venustum H.Koidz, Taraxacum hondoense Nakai, Taraxacum platycarpum Dahlst, Taraxacum japonicum, Taraxacum officinale Weber, and Taraxacum koksaghyz. Preferably, it is a plant belonging to the family Euphorbiaceae, e.g. the genus Hevea, more preferably a plant belonging to the genus Hevea. The rubber-producing plant is particularly preferably Hevea brasiliensis, among others.

The steps in the production method of the present invention are described in the following. The description that follows uses Hevea brasiliensis as an example of the rubber-producing plant, but the production method of the present invention can also be carried out in the same manner for rubber-producing plants other than Hevea brasiliensis.

### (Culture Step)

The culture step includes culturing a tissue fragment derived from a rubber-producing plant to obtain a cultured tissue fragment. This step is not particularly limited and may utilize methods commonly used to culture a tissue fragment derived from a rubber-producing plant to obtain a cultured tissue fragment. However, the culture step is, for example, preferably an induction step of culturing a Hevea brasiliensis-derived tissue containing a node, axillary bud, or apical bud in an induction medium containing a plant growth hormone and a carbon source to induce and form a cultured tissue fragment, preferably a shoot.

The cultured tissue fragment may be, for example, a shoot or node, and is preferably a shoot because this allows for a more efficient and more stable execution of the subsequent steps for producing a transgenic plant. The following mainly describes the case where the cultured tissue fragment is a shoot, but the production method of the present invention can also be carried out in the same manner for cultured tissue fragments other than shoots.

The source material (tissue) for inducing the shoot may be a Hevea brasiliensis tissue containing a node, axillary bud, or apical bud, and specifically, for example, a tissue containing a node, axillary bud, or apical bud and derived from a mature or young tree, sapling or clone plantlet, or from an aseptic seedling grown in vitro from a seed (aseptic seedling). When the tissue is a tissue containing a node, axillary bud, or apical bud and derived from a mature or young tree, sapling or clone plantlet, it may be cut to the required size as appropriate followed by disinfection or sterilization of the surface before use; while when the tissue is a tissue containing a node, axillary bud, or apical bud and derived from an aseptic seedling grown in vitro from a seed (aseptic seedling), it may be cut to the required size as appropriate before use.

In the case of the tissue containing a node, axillary bud, or apical bud and derived from a mature or young tree, sapling or clone plantlet, the surface of the tissue is first cleaned prior to culture in the induction medium. For example, cleaning may be carried out with an abrasive powder or a soft sponge, but cleaning with running water is preferred. The water used for cleaning may contain approximately 0.1 mass % of a surfactant.

The tissue is then disinfected or sterilized. The disinfection or sterilization may be carried out using known disinfectants or sterilizing agents, but ethanol, benzalkonium chloride, and an aqueous sodium hypochlorite solution are preferred. An additional washing with sterile water may be performed after the disinfection or sterilization treatment.

For example, the following procedure is a specific example of the cleaning and disinfection or sterilization treatment: clean the tissue surface with running water; then wash it with ethanol; then sterilize it using an aqueous sodium hypochlorite solution, optionally while stirring; and then wash it with sterile water.

The induction step includes culturing a Hevea brasiliensis tissue containing a node, axillary bud, or apical bud in an induction medium containing a plant growth hormone and a carbon source to induce and form a shoot. The induction medium may be a liquid or a solid, but solid culture is preferred because shoot induction is facilitated by culture of the tissue inserted in the medium. When the induction medium is a liquid medium, static culture or shake culture may be carried out.

When the disinfected or sterilized tissue is used, the cut end may be cut off in order to eliminate the effect of the disinfectant or sterilizing agent prior to culture.

Examples of the plant growth hormone include auxin plant hormones and/or cytokinin plant hormones. Among these, cytokinin plant hormones are preferred.

The auxin plant hormones can be exemplified by 2,4-dichlorophenoxyacetic acid, 1-naphthaleneacetic acid, indole-3-butyric acid, indole-3-acetic acid, indolepropionic acid, chlorophenoxyacetic acid, naphthoxyacetic acid, phenylacetic acid, 2,4,5-trichlorophenoxyacetic acid, para-chlorophenoxyacetic acid, 2-methyl-4-chlorophenoxyacetic acid, 4-fluorophenoxyacetic acid, 2-methoxy-3,6-dichlorobenzoic acid, 2-phenyl acid, picloram, and picolinic acid. Among the foregoing, 2,4-dichlorophenoxyacetic acid, 1-naphthaleneacetic acid, and indole-3-butyric acid are preferred, with 2,4-dichlorophenoxyacetic acid or 1-naphthaleneacetic acid being more preferred.

The cytokinin plant hormones can be exemplified by benzyladenine, kinetin, zeatin, benzylaminopurine, isopentynylaminopurine, thidiazuron, isopentenyladenine, zeatin riboside, and dihydrozeatin. Among the foregoing, benzyladenine, kinetin, and zeatin are preferred; benzyladenine or kinetin is more preferred; and benzyladenine is still more preferred.

There are no particular limitations on the carbon source, and examples include sugars such as sucrose, glucose, trehalose, fructose, lactose, galactose, xylose, allose, talose, gulose, altrose, mannose, idose, arabinose, apiose, maltose, and so forth. Sucrose is preferred among the foregoing.

The induction medium preferably further contains active carbon in order to prevent growth inhibitors from accumulating in the tissue. The induction medium also preferably further contains silver nitrate in order to promote shoot formation. The induction medium may also contain coconut water (coconut milk) in order to promote shoot formation.

The following base media can be used as the induction medium: basal media such as White's medium (disclosed on pp. 20-36 of Shokubutsu Saibo Kogaku Nyumon (Introduction to Plant Cell Engineering), Japan Scientific Societies Press), Heller's medium (Heller R, Bot. Biol. Veg. Paris 14, 1-223 (1953)), SH medium (Schenk and Hildebrandt medium), MS medium (Murashige and Skoog medium) (disclosed on pp. 20-36 of Shokubutsu Saibo Kogaku Nyumon (Introduction to Plant Cell Engineering), Japan Scientific Societies Press), LS medium (Linsmaier and Skoog medium) (disclosed on pp. 20-36 of Shokubutsu Saibo Kogaku Nyumon (Introduction to Plant Cell Engineering), Japan Scientific Societies Press), Gamborg medium, B5 medium (disclosed on pp. 20-36 of Shokubutsu Saibo Kogaku Nyumon (Introduction to Plant Cell Engineering), Japan Scientific Societies Press), MB medium, and WP medium (Woody Plant: for woody plants)(the disclosures of the foregoing documents are incorporated by reference herein), and modified basal media obtained by altering the composition of the basal media, and so forth. Among the foregoing, MS medium and modified MS media obtained by altering the composition of MS medium are preferred.

When the induction medium is used as a solid medium, the medium may be converted into a solid using a solidifying agent. There are no particular limitations on the solidifying agent, and examples include agar, gellan gum (e.g. Gelrite, Phytagel), agarose, and so forth.

The suitable composition and culture conditions of the induction medium vary depending on the type of plant and also vary depending on whether the medium is a liquid medium or a solid medium, but the composition is usually as follows, particularly in the case of Hevea brasiliensis.

The carbon source concentration in the induction medium is preferably at least 0.1 mass %, more preferably at least 1.0 mass %. The carbon source concentration is preferably not more than 10 mass %, more preferably not more than 5.0 mass %. In the specification, the carbon source concentration denotes the sugar concentration.

Preferably, substantially no auxin plant hormone is added to the induction medium, and the auxin plant hormone concentration in the induction medium is in particular preferably not more than 1.0 mg/L, more preferably not more than 0.1 mg/L, still more preferably not more than 0.05 mg/L, particularly preferably not more than 0.01 mg/L.

When a cytokinin plant hormone is added to the induction medium, the cytokinin plant hormone concentration in the induction medium is preferably at least 0.01 mg/L, more preferably at least 0.1 mg/L, still more preferably at least 0.5 mg/L, and particularly preferably at least 0.8 mg/L. The cytokinin plant hormone concentration is preferably not more than 7.0 mg/L, more preferably not more than 6.0 mg/L.

Particularly when benzyladenine is used as the cytokinin plant hormone, the benzyladenine concentration is preferably 4.0 to 6.0 mg/L, and most preferably 5.0 mg/L. When, on the other hand, kinetin is used as the cytokinin plant hormone, the kinetin concentration is preferably 0.8 to 1.2 mg/L, and most preferably 1.0 mg/L.

The active carbon concentration in the induction medium is preferably at least 0.01 mass %, more preferably at least 0.03 mass %. The active carbon concentration is preferably not more than 1.0 mass %, more preferably not more than 0.1 mass %.

The silver nitrate concentration in the induction medium is preferably at least 0.1 mg/L, more preferably at least 0.3 mg/L, still more preferably at least 0.5 mg/L. The silver nitrate concentration is preferably not more than 5.0 mg/L, more preferably not more than 3.0 mg/L.

The pH of the induction medium is preferably 4.0 to 10.0, more preferably 5.0 to 6.5, still more preferably 5.5 to 6.0.

In the specification, the pH of the solid medium denotes the pH of the medium that incorporates all the components except the solidifying agent.

The induction step is usually carried out in a controlled environment in which culture conditions such as temperature, light cycle, and so forth are managed. The culture conditions may be selected as appropriate, but, for example, the culture temperature is preferably 0° C. to 40° C., more preferably 20° C. to 40° C., still more preferably 25° C. to 35° C. Culture may be carried out in the dark or in the light, and the light conditions may be, for example, under a 14-16 h light cycle at 12.5 μmol/m²/s. There are no particular limitations on the culture time, but culture for 1 to 10 weeks is preferred, and culture for 4 to 8 weeks is more preferred.

When the induction medium is a solid medium, the solidifying agent concentration in the induction medium is preferably at least 0.1 mass %, more preferably at least 0.2 mass %, still more preferably at least 0.5 mass %. The solidifying agent concentration is preferably not more than 2.0 mass %, more preferably not more than 1.1 mass %, still more preferably not more than 0.8 mass %.

Among the conditions indicated above, it is particularly preferred that the plant growth hormone is a cytokinin plant hormone, particularly benzyladenine or kinetin, at a concentration of 0.8 to 6.0 mg/L, and the culture temperature is 25° C. to 35° C.

As described above, a shoot can be induced and formed by culturing in the induction medium a Hevea brasiliensis tissue containing a node, axillary bud, or apical bud.

The shoot formed in the induction step (culture step) is subjected to a transformation step, which will be described later; but by subjecting the shoot to a propagation step, which will be described later, the number of shoots can be increased and the tissue to be used as a graft can be mass propagated. The mass propagated graft tissue obtained in such a propagation step may also be subjected to the transformation step described later. In this case, the number of shoots can be increased and the graft tissue can be mass propagated. Furthermore, further mass propagation can be carried out by repeating such a propagation step and/or by subculture. Thus, the culture step may further be followed by the propagation step prior to the transformation step, i.e., the shoot propagated by the so-called micropropagation method may be subjected to the transformation step. Thus, in another suitable embodiment of the present invention, the production method of the present invention further includes, after the culture step, a propagation step of collecting and subdividing the cultured tissue fragment obtained in the culture step, and culturing the subdivided cultured tissue fragment in an induction medium containing a plant growth hormone and a carbon source to obtain a cultured tissue fragment, and the cultured tissue fragment obtained in the propagation step is subjected to the transformation step.

Here, the shoot formed in the induction step can, once stable shoot growth has been confirmed, be subjected to the transformation step or the propagation step. For example, when culture is carried out for 4 weeks in the induction medium, the shoot is not only induced but undergoes elongation, and not simply the induced shoot but also the induced and elongated shoot may then be subjected to the transformation step or propagation step. The degree of shoot elongation at this point can be controlled as appropriate by culture conditions in the induction medium, e.g. culture time. In addition, the shoot induced in the induction step may be subjected to the transformation step or propagation step after it has been elongated by the elongation step described below.

### (Elongation Step)

The elongation step includes culturing the shoot formed in the induction step in an elongation medium containing a plant growth hormone and a carbon source to elongate the shoot. Specifically, the shoot (e.g. of approximately 2 to 3 cm) formed in the induction step is transferred by insertion into the elongation medium and cultured for approximately 4 weeks, so that the shoot elongates and new buds can also be acquired.

The elongation medium may be a liquid or a solid, but solid culture is preferred because shoot elongation is facilitated by culture of the shoot inserted in the medium. When the elongation medium is a liquid medium, static culture or shake culture may be carried out.

The elongation medium contains a plant growth hormone and a carbon source, and examples of the plant growth hormone include auxin plant hormones and/or cytokinin plant hormones. In particular, combinations of auxin plant hormones and cytokinin plant hormones are preferred.

The auxin plant hormones as described for the induction medium may be used here as the auxin plant hormone, but 2,4-dichlorophenoxyacetic acid, 1-naphthaleneacetic acid, and indole-3-butyric acid, among others, are preferred; 2,4-dichlorophenoxyacetic acid or 1-naphthaleneacetic acid is more preferred; and 1-naphthaleneacetic acid is particularly preferred.

The cytokinin plant hormones as described for the induction medium may be used here as the cytokinin plant hormone, but benzyladenine, kinetin, and zeatin, among others, are preferred; benzyladenine or kinetin is more preferred; and benzyladenine is still more preferred.

The carbon source used in the elongation medium is not particularly limited and the carbon sources as described for the induction medium may be used here, among which sucrose is preferred.

The elongation medium preferably further contains active carbon or silver nitrate as described for the induction medium.

Base media such as the basal media as described for the induction medium, and modified basal media obtained by altering the composition of the basal media may be used as the elongation medium, but MS medium, B5 medium, and WP medium, among others, are preferred, and MS medium and modified MS media obtained by altering the composition of MS medium are more preferred.

When the elongation medium is used as a solid medium, the medium may be converted into a solid using a solidifying agent. There are no particular limitations on the solidifying agent, and examples include agar, gellan gum (e.g. Gelrite, Phytagel), agarose, and so forth.

The suitable composition and culture conditions of the elongation medium vary depending on the type of plant and also vary depending on whether the medium is a liquid medium or a solid medium, but the composition is usually as follows, particularly in the case of Hevea brasiliensis.

The carbon source concentration in the elongation medium is preferably at least 0.1 mass %, more preferably at least 1.0 mass %. The carbon source concentration is preferably not more than 10 mass %, more preferably not more than 5.0 mass %.

When an auxin plant hormone is added to the elongation medium, the auxin plant hormone concentration in the elongation medium is preferably at least 0.01 mg/L, more preferably at least 0.03 mg/L, still more preferably at least 0.05 mg/L. The auxin plant hormone concentration is preferably not more than 2.0 mg/L, more preferably not more than 1.0 mg/L, still more preferably not more than 0.1 mg/L, particularly preferably not more than 0.08 mg/L.

When a cytokinin plant hormone is added to the elongation medium, the cytokinin plant hormone concentration in the elongation medium is preferably at least 0.01 mg/L, more preferably at least 0.1 mg/L, still more preferably at least 0.5 mg/L, particularly preferably at least 0.8 mg/L. The cytokinin plant hormone concentration is preferably not more than 5.0 mg/L, more preferably not more than 2.0 mg/L.

The active carbon concentration in the elongation medium is preferably at least 0.01 mass %, more preferably at least 0.03 mass %. The active carbon concentration is preferably not more than 1.0 mass %, more preferably not more than 0.1 mass %.

The silver nitrate concentration in the elongation medium is preferably at least 0.1 mg/L, more preferably at least 0.3 mg/L, still more preferably at least 0.5 mg/L. The silver nitrate concentration is preferably not more than 5.0 mg/L, more preferably not more than 3.0 mg/L.

The pH of the elongation medium is preferably 4.0 to 10.0, more preferably 5.0 to 6.5, still more preferably 5.5 to 6.0.

The elongation step is usually carried out in a controlled environment in which culture conditions such as temperature, light cycle, and so forth are managed. The culture conditions may be selected as appropriate, but, for example, the culture temperature is preferably 0° C. to 40° C., more preferably 20° C. to 40° C., still more preferably 25° C. to 35° C. Culture may be carried out in the dark or in the light, and the light conditions may be, for example, under a 14-16 h light cycle at 12.5 μmol/m²/s. There are no particular limitations on the culture time, but culture for 1 to 10 weeks is preferred, and culture for 3 to 5 weeks is more preferred.

When the elongation medium is a solid medium, the solidifying agent concentration in the elongation medium is preferably at least 0.1 mass %, more preferably at least 0.2 mass %, still more preferably at least 0.5 mass %. The solidifying agent concentration is preferably not more than 2.0 mass %, more preferably not more than 1.1 mass %, still more preferably not more than 0.8 mass %.

Among the conditions indicated above, it is particularly preferred that the plant growth hormone is an auxin plant hormone, particularly 1-naphthaleneacetic acid, plus a cytokinin plant hormone, particularly benzyladenine, at concentrations of 0.05 to 0.08 mg/L and 0.8 to 2.0 mg/L, respectively, and the culture temperature is 25° C. to 35° C.

As described above, shoot elongation can be carried out by culturing in the elongation medium the shoot formed in the induction step. Further, not only does the shoot elongate but new shoots are also formed in this elongation step. The shoot elongated in the elongation step can further be used in the transformation step or propagation step described below.

### (Propagation Step)

The propagation step includes collecting and subdividing the shoot formed in the induction step or the shoot elongated in the elongation step, and culturing the subdivided shoot in an induction medium containing a plant growth hormone and a carbon source to induce shoot formation. Through this step, the number of shoots can be increased and the tissue to be used as a graft can be mass propagated. Furthermore, further mass propagation can be carried out by repeating this step and/or by subculture.

Advantageously, a stable increase in the number of shoots can be achieved in the propagation step when the shoot formed in the induction step or the shoot elongated in the elongation step is collected so as not to collect any portion containing a node, axillary bud, or apical bud.

The collected shoot can be subdivided by conventional methods, and the size of subdivision may be selected as appropriate.

The induction media as described for the induction step may be used as the induction medium used in the propagation step.

As described above, the shoot can be mass propagated in the propagation step by collecting and subdividing the shoot and culturing the subdivided shoot in the induction medium to induce shoot formation. The shoot formed in this propagation step is subjected to the transformation step described below. The shoot can be subjected to the transformation step once stable shoot growth can be confirmed; however, prior to the transformation step, the formed shoot may be subcultured and grown in the induction medium and then subjected to the transformation step, or may be subjected to the aforementioned elongation step followed by the transformation step.

### (Transformation Step)

The transformation step includes transforming with a gene construct the cultured tissue fragment obtained in the culture step or the cultured tissue fragment obtained in the propagation step, and specifically the shoot formed in the induction step or the shoot formed in the propagation step.

A method of indirectly introducing a gene into a plant using Agrobacterium is used as the transformation method here.

Specifically, this transformation step includes an infection step of culturing the cultured tissue fragment obtained in the culture step, or the cultured tissue fragment obtained in the propagation step, in the presence of an Agrobacterium that has been transformed with a gene construct, and a decontamination step of culturing the cultured tissue fragment obtained in the infection step in a decontamination medium containing a carbapenem antibiotic to decontaminate the Agrobacterium.

In the infection step, the shoot formed in the induction step or the shoot formed in the propagation step is infected by an Agrobacterium containing a gene construct with a target gene or fragment thereof (also referred to hereinafter as “target gene or the like” or as “genetic material”). The method for preparing the Agrobacterium (Agrobacterium preparation step) is described first.

### (Agrobacterium Preparation Step)

The Agrobacterium used in the infection step is not particularly limited as long as it is an Agrobacterium that can introduce the gene construct that it contains into the plant cells; however, it is preferably Agrobacterium tumefaciens.

This is because it has good infection efficiency and is generally used in the Agrobacterium method.

The Agrobacterium containing a gene construct with a target gene or the like may be prepared by any conventional techniques. An example is a method of incorporating a target gene or the like into a plasmid capable of homologous recombination with the T-DNA region of the Ti plasmid present in Agrobacterium to construct a target gene-recombined intermediate vector, and introducing the target gene-recombined intermediate vector into an Agrobacterium to prepare an Agrobacterium containing a gene construct with the target gene or the like. Another example is a method of incorporating a target gene or the like into a binary vector, which is generally used in the Agrobacterium method, to construct a target gene binary vector, and introducing the target gene binary vector into an Agrobacterium to prepare an Agrobacterium containing a gene construct with the target gene or the like.

The target gene present in the gene construct denotes a gene that is intended to be introduced into a target rubber-producing plant (target plant). There are no particular limitations on the target gene as long as the genetic traits of a target plant can be modified as a result of the introduction of the target gene into the target plant. The target gene may be a gene originally possessed by a target plant into which it is to be introduced, or a gene derived from an organism other than the target plant, or an artificially constructed gene. The artificially constructed gene may be, for example, a chimeric gene in which two or more genes are linked, or a mutant gene formed by mutation of a gene of any organism. The mutant gene may be formed, for example, by partial deletion or substitution of the bases in the DNA nucleotide sequence of a gene. Or, the mutant gene may be formed by insertion of a partial nucleotide sequence within the nucleotide sequence.

Thus, in another suitable embodiment of the present invention, the gene construct contains genetic material that is homologous to the genome of the target rubber-producing plant. Still another suitable embodiment of the present invention is that the gene construct contains genetic material that is heterologous to the genome of the target rubber-producing plant.

The target gene may be a structural gene or a regulatory region. For example, it may be a structural gene that contains a transcription or translation control region, e.g. a promoter or terminator. It goes without saying that the gene of the control region may be any gene that can function in a target plant into which the gene is to be introduced, and may be a gene derived from an organism of the same species as the target plant into which the gene is to be introduced or a gene derived from an organism of a different species. Examples of such heterologous promoters include promoters generally used in fields related to genetic recombination, such as CaMV35 promoter, NOS promoter, and so forth.

The target gene to be introduced into a target plant may be a full-length gene or a fragment thereof. For example, a fragment consisting only of a functional domain of a structural gene may be introduced.

The target gene to be introduced into a target plant may be any reporter gene, which will be described later, or any gene that produces a desired effect in the cells of the target plant. The desired effect may be any change, for example, growth acceleration, disease resistance, or alteration or improvement in the quality of a plant product.

When a regulatory region (e.g. a promoter) that functions in a tissue-specific manner is incorporated into such a gene, it is possible to express the protein encoded by the target gene in a specific tissue of the plant.

The target gene or the like in the gene construct is suitably incorporated into a vector along with a marker gene and optionally a reporter gene.

The marker gene (also referred to as selective marker gene) may be any gene that codes for a selective marker that confers resistance to a selective reagent present in a selective culture medium, which will be described later, and examples include drug resistance genes such as kanamycin-resistance gene (nptII), hygromycin-resistance gene (hptI), glyphosate-resistance gene, bleomycin-resistance gene, and paromomycin-resistance gene. Examples of the reporter gene for determining the expression site in the plant include luciferase gene, GUS (β-glucuronidase) gene, green fluorescent protein (GFP) gene, and red fluorescent protein (RFP) gene.

Thus, in another suitable embodiment of the present invention, the gene construct contains a selective marker gene that confers resistance to a selective reagent.

In the Agrobacterium preparation step, the Agrobacterium containing a gene construct with a target gene or the like, prepared, for example, as described above can be cultured (for example, shake cultured for 10 to 30 hours in YEB medium at a culture temperature of 20° C. to 35° C.) and propagated by usual methods to prepare an amount required to infect the shoot derived from a target plant.

### (Infection Step)

In the infection step, the shoot formed in the induction step or the shoot formed in the propagation step is infected with the Agrobacterium containing a gene construct with a target gene or the like (i.e., the Agrobacterium obtained in the Agrobacterium preparation step).

The infection step can be carried out by procedures commonly used in the Agrobacterium method. For example, infection may be carried out by suspending the Agrobacterium in a liquid infection medium and immersing in the suspension the shoot formed in the induction step or the shoot formed in the propagation step. After the immersion, the shoot may be separated from the suspension using, for example, filter paper. The shoot may be immersed under static or shaking conditions, but it is preferably immersed with shaking because this facilitates infection of the shoot by the Agrobacterium.

The bacterial concentration in the Agrobacterium suspension used for infection may be selected as appropriate in view of, for example, the type and growth activity of the Agrobacterium, the culture time after shoot induction of the shoot to be infected, the immersion time, and so forth. For example, an Agrobacterium population corresponding to 10 to 50 mL, preferably 20 to 40 mL, more preferably 25 to 35 mL, of an Agrobacterium suspension having an absorbance measured at 600 nm (O.D. 600) of 0.01 to 1.0, preferably 0.05 to 0.8, more preferably O. 08 to 0.6, is preferably brought into contact with five shoots. This can optimize the number of Agrobacterium cells that infect the shoot, thereby making it possible to efficiently produce the transformed shoot.

The Agrobacterium/shoot coexistence time in the infection step, i.e., the time during which the shoot is in contact with the Agrobacterium, is preferably 0.5 to 60 minutes, more preferably 1 to 40 minutes, still more preferably 25 to 35 minutes. This can optimize the number of Agrobacterium cells that infect the shoot, thereby making it possible to efficiently produce the transformed shoot. The coexistence time refers to, for example, the immersion time when the shoot is immersed in the Agrobacterium suspension.

The infection medium for suspending the Agrobacterium may be any base medium such as any of the basal media as described above, and modified basal media obtained by altering the composition of the basal media, optionally supplemented with a plant growth hormone and a carbon source. Preferred thereamong are MS medium, LS medium, B5 medium, and WP medium, with MS medium being more preferred. The plant growth hormones and carbon sources as described for the induction medium can be suitably used here. The carbon source may be a combination of sucrose and glucose.

The infection medium preferably further contains silver nitrate as described for the induction medium.

The suitable composition of the infection medium varies depending on the type of plant, but the composition is usually as follows, particularly in the case of Hevea brasiliensis.

The carbon source concentration in the infection medium is preferably at least 0.1 mass %, more preferably at least 1 mass %, still more preferably at least 2 mass %, particularly preferably at least 3 mass %. The carbon source concentration is preferably not more than 10 mass %, more preferably not more than 6 mass %, still more preferably not more than 4 mass %.

Preferably, substantially no auxin plant hormone is added to the infection medium, and the auxin plant hormone concentration in the infection medium is in particular preferably not more than 1.0 mg/L, more preferably not more than 0.1 mg/L, still more preferably not more than 0.05 mg/L, particularly preferably not more than 0.01 mg/L.

When a cytokinin plant hormone is added to the infection medium, the cytokinin plant hormone concentration in the infection medium is preferably at least 0.01 mg/L, more preferably at least 0.1 mg/L, still more preferably at least 0.5 mg/L, and particularly preferably at least 0.8 mg/L. The cytokinin plant hormone concentration is preferably not more than 7.0 mg/L, more preferably not more than 6.0 mg/L. Particularly when benzyladenine is used as the cytokinin plant hormone, the benzyladenine concentration is preferably 4.0 to 6.0 mg/L, and most preferably 5.0 mg/L. When, on the other hand, kinetin is used as the cytokinin plant hormone, the kinetin concentration is preferably 0.8 to 1.2 mg/L, and most preferably 1.0 mg/L.

The silver nitrate concentration in the infection medium is preferably at least 0.1 mg/L, more preferably at least 0.3 mg/L, still more preferably at least 0.5 mg/L. The silver nitrate concentration is preferably not more than 5.0 mg/L, more preferably not more than 3.0 mg/L.

In another preferred embodiment, the infection medium further contains acetosyringone, i.e., it is an acetosyringone-containing medium because the shoot is well infected with Agrobacterium. When acetosyringone is added to the infection medium, the acetosyringone concentration in the infection medium is preferably 1 to 500 μM, more preferably 10 to 400 μM, still more preferably 50 to 250 μM.

The pH of the infection medium is not particularly limited, but is preferably 4.0 to 10.0, more preferably 5.0 to 6.0. The infection temperature (the temperature of the infection medium) is preferably 0° C. to 40° C., more preferably 20° C. to 36° C., still more preferably 22° C. to 30° C., and most preferably 28° C. The infection step may be carried out in the dark or in the light.

Among the conditions indicated above, it is particularly preferred that the plant growth hormone is a cytokinin plant hormone, particularly benzyladenine, at a concentration of 4.0 to 6.0 mg/L, and the culture temperature is 22° C. to 30° C.

As described above, the shoot can be infected with the Agrobacterium in the infection step, for example, by suspending the Agrobacterium obtained in the Agrobacterium preparation step in the liquid infection medium and immersing in the suspension the shoot formed by the induction step or the shoot formed in the propagation step. After the immersion, the shoot is separated from the suspension using, for example, filter paper, and the separated shoot is then subjected to a decontamination step in which the Agrobacterium is decontaminated. However, it is preferably subjected to the co-culture step described below prior to the decontamination step. Thus, in the transformation step, the co-culture step is preferably performed after the infection step and prior to the decontamination step.

### (Co-Culture Step)

The co-culture step includes culturing the shoot obtained in the infection step (the shoot infected with the Agrobacterium) in a co-culture medium. As a result, the gene fragment (target gene or the like) that has been introduced into the shoot by infection can be incorporated into the genes of the plant cells to obtain a more stably transformed shoot.

The co-culture medium may be a liquid or a solid, but solid culture is preferred because a stably transformed shoot can be obtained by plating and culturing on the medium. When the co-culture medium is a liquid medium, static culture or shake culture may be carried out.

The co-culture medium may be any base medium such as any of the basal media as described above, and modified basal media obtained by altering the composition of the basal media, optionally supplemented with a plant growth hormone and a carbon source. MS medium, B5 medium, and WP medium, among others, are preferred, and MS medium and modified MS media obtained by altering the composition of MS medium are more preferred. The plant growth hormones and carbon sources as described for the induction medium can be suitably used here.

The co-culture medium preferably further contains silver nitrate as described for the induction medium.

The suitable composition of the co-culture medium varies depending on the type of plant, but the composition is usually as follows, particularly in the case of Hevea brasiliensis.

The carbon source concentration in the co-culture medium is preferably at least 0.1 mass %, more preferably at least 1 mass %, still more preferably at least 2 mass %, particularly preferably at least 3 mass %. The carbon source concentration is preferably not more than 10 mass %, more preferably not more than 6 mass %, still more preferably not more than 4 mass %.

Preferably, substantially no auxin plant hormone is added to the co-culture medium, and the auxin plant hormone concentration in the co-culture medium is in particular preferably not more than 1.0 mg/L, more preferably not more than 0.1 mg/L, still more preferably not more than 0.05 mg/L, particularly preferably not more than 0.01 mg/L.

When a cytokinin plant hormone is added to the co-culture medium, the cytokinin plant hormone concentration in the co-culture medium is preferably at least 0.01 mg/L, more preferably at least 0.1 mg/L, still more preferably at least 0.5 mg/L, and particularly preferably at least 0.8 mg/L. The cytokinin plant hormone concentration is preferably not more than 7.0 mg/L, more preferably not more than 6.0 mg/L.

Particularly when benzyladenine is used as the cytokinin plant hormone, the benzyladenine concentration is preferably 4.0 to 6.0 mg/L, and most preferably 5.0 mg/L. When, on the other hand, kinetin is used as the cytokinin plant hormone, the kinetin concentration is preferably 0.8 to 1.2 mg/L, and most preferably 1.0 mg/L.

The silver nitrate concentration in the co-culture medium is preferably at least 0.1 mg/L, more preferably at least 0.3 mg/L, still more preferably at least 0.5 mg/L. The silver nitrate concentration is preferably not more than 5.0 mg/L, more preferably not more than 3.0 mg/L.

Preferably, the co-culture medium further contains acetosyringone, i.e., it is an acetosyringone-containing medium because a stably transformed shoot is then more easily produced. The acetosyringone concentration in the co-culture medium is preferably 1 to 500 μM, more preferably 10 to 400 μM, still more preferably 50 to 250 μM.

When the co-culture medium is used as a solid medium, the medium may be converted into a solid using a solidifying agent as described for the induction medium.

When the co-culture medium is a solid medium, the solidifying agent concentration in the co-culture medium is preferably at least 0.1 mass %, more preferably at least 0.2 mass %, still more preferably at least 0.5 mass %. The solidifying agent concentration is preferably not more than 2 mass %, more preferably not more than 1.1 mass %, still more preferably not more than 0.8 mass %.

The pH of the co-culture medium is not particularly limited, but is preferably 4.0 to 10.0, more preferably 5.0 to 6.0.

The culture temperature is preferably 0° C. to 40° C., more preferably 10° C. to 36° C., still more preferably 20° C. to 28° C. Culture may be carried out in the dark or in the light, but is preferably carried out in the dark where the illuminance is preferably 0 to 0.1 lx. The culture time is not particularly limited, but culture for 2 to 4 days is preferred.

Among the conditions indicated above, it is particularly preferred that the plant growth hormone is a cytokinin plant hormone, particularly benzyladenine, at a concentration of 4.0 to 6.0 mg/L, and the culture temperature is 20° C. to 28° C.

As described above, according to the co-culture step, by culturing in the co-culture medium the shoot obtained in the infection step (the shoot infected with the Agrobacterium), the gene fragment (target gene or the like) that has been introduced into the shoot by infection can be incorporated into the genes of the plant cells to obtain a more stably transformed shoot. Preferably, the shoot obtained in the co-culture step (a mixture of transformed and untransformed shoots) is first subjected to a subsequent decontamination step and then to a subsequent selective culture step.

### (Decontamination Step)

The decontamination step includes culturing the cultured tissue fragment obtained in the infection step, or the cultured tissue fragment obtained in the co-culture step, in a decontamination medium containing a carbapenem antibiotic to decontaminate the Agrobacterium. This step can decontaminate the Agrobacterium present with the shoot obtained in the infection step or the shoot obtained in the co-culture step. While this step can be carried out by the procedures commonly used in the Agrobacterium method, the present invention is characterized particularly by using a carbapenem antibiotic as the decontaminating agent to decontaminate the Agrobacterium. Due to this feature it is possible to reduce the concentration of the decontaminating agent used to decontaminate the Agrobacterium to low levels and to thoroughly decontaminate the Agrobacterium in a short period of time. It is therefore possible to minimize the adverse effects on the plant arising from decontamination of the Agrobacterium to prevent a decline in the survival rate of the transformed tissue fragment, thereby increasing the efficiency of producing the genetically modified transgenic plant. Thus, a genetically modified transgenic plant can be efficiently produced in a short period of time.

The decontamination step is not particularly limited as long as it can decontaminate and remove the Agrobacterium present with the shoot obtained in the infection step or the shoot obtained in the co-culture step (a mixture of transformed and untransformed shoots) using a carbapenem antibiotic. For example, the Agrobacterium may be decontaminated by washing the shoot obtained in the infection step, or the shoot obtained in the co-culture step, with a liquid decontamination medium followed by culture in a decontamination medium. Specifically, the decontamination step may be carried out by: washing the shoot obtained in the co-culture step by immersion in a liquid decontamination medium; after the immersion, separating the shoot from the liquid decontamination medium using, for example, filter paper; and culturing the separated shoot in a decontamination medium.

The shoot may be immersed in the liquid decontamination medium under static or shaking conditions. Moreover, washing with the liquid decontamination medium may be performed once or may be repeated a plurality of times.

The liquid decontamination medium may be prepared by adding a carbapenem antibiotic, and optionally a plant growth hormone and a carbon source, to any base medium such as any of the basal media as described above, and modified basal media obtained by altering the composition of the basal media. Specifically, the liquid decontamination medium may suitably be prepared by adding a carbapenem antibiotic to the same medium as the infection medium.

The liquid decontamination medium preferably further contains silver nitrate as described for the infection medium.

The carbapenem antibiotic refers to a β-lactam antibiotic having a β-lactam ring in which a carbon atom is substituted for the sulfur atom bonded to the β-lactam ring, which is generally present in other β-lactam antibiotics.

Specific examples of the carbapenem antibiotic include imipenem, meropenem, and doripenem, with meropenem being particularly preferred because then the effects of the present invention can be more suitably achieved. Each of these carbapenem antibiotics may be used alone, or two or more of these may be used in combination.

The carbapenem antibiotic concentration in the liquid decontamination medium is preferably 10 to 500 mg/L. Due to the use of a carbapenem antibiotic as the decontaminating agent, the Agrobacterium can be thoroughly decontaminated by washing with the liquid decontamination medium, even when the concentration of the decontaminating agent used for decontamination of the Agrobacterium is reduced to low levels as indicated above, and therefore the adverse effects on the plant arising from decontamination of the Agrobacterium can be minimized to prevent a decline in the survival rate of the transformed tissue fragment. The carbapenem antibiotic concentration is more preferably at least 12.5 mg/L, still more preferably at least 20 mg/L, further preferably at least 30 mg/L. It is also more preferably not more than 250 mg/L, still more preferably not more than 125 mg/L, further preferably not more than 100 mg/L, particularly preferably not more than 70 mg/L. A thorough decontamination of the Agrobacterium may not be achieved at less than 10 mg/L. Also, at above 500 mg/L the carbapenem antibiotic concentration is too high and can cause adverse effects not only on the Agrobacterium but even on shoot survival.

In addition to the carbapenem antibiotic, additional decontaminating agents may also be used in the liquid decontamination medium as long as the effects of the present invention are not impaired.

These additional decontaminating agents can be exemplified by cefotaxime, carbenicillin, augmentin, and so forth.

The duration of washing with the liquid decontamination medium, i.e., the duration during which the shoot is in contact with the liquid decontamination medium, is preferably 0.5 to 60 minutes. Due to the use of a carbapenem antibiotic as the decontaminating agent, the Agrobacterium can be thoroughly decontaminated using a short period of time as indicated above as the duration of washing with the liquid decontamination medium, even when the carbapenem antibiotic used is at a low concentration as indicated above, and therefore the adverse effects on the plant arising from decontamination of the Agrobacterium can be minimized to prevent a decline in the survival rate of the transformed tissue fragment. The washing duration is more preferably 1 to 40 minutes, still more preferably 5 to 30 minutes.

The washing duration denotes, for example, the immersion time when the shoot is immersed in the liquid decontamination medium.

In addition, if washing with the liquid decontamination medium is repeated a plurality of times, the washing duration then represents the total of the washing durations (total washing duration).

The suitable composition of the liquid decontamination medium varies depending on the type of plant, but the composition is usually as follows, particularly in the case of Hevea brasiliensis.

The carbon source concentration in the liquid decontamination medium is preferably at least 0.1 mass %, more preferably at least 1 mass %, still more preferably at least 2 mass %, particularly preferably at least 3 mass %. The carbon source concentration is preferably not more than 10 mass %, more preferably not more than 6 mass %, still more preferably not more than 5 mass %, further preferably not more than 4 mass %.

Preferably, substantially no auxin plant hormone is added to the liquid decontamination medium, and the auxin plant hormone concentration in the liquid decontamination medium is in particular preferably not more than 1.0 mg/L, more preferably not more than 0.1 mg/L, still more preferably not more than 0.05 mg/L, particularly preferably not more than 0.01 mg/L.

When a cytokinin plant hormone is added to the liquid decontamination medium, the cytokinin plant hormone concentration in the liquid decontamination medium is preferably at least 0.01 mg/L, more preferably at least O. 1 mg/L, still more preferably at least 0.5 mg/L, further preferably at least 0.8 mg/L, particularly preferably at least 1.0 mg/L, most preferably at least 3.0 mg/L. The cytokinin plant hormone concentration is preferably not more than 7.0 mg/L, more preferably not more than 6.0 mg/L.

Particularly when benzyladenine is used as the cytokinin plant hormone, the benzyladenine concentration is preferably 4.0 to 6.0 mg/L, and most preferably 5.0 mg/L. When, on the other hand, kinetin is used as the cytokinin plant hormone, the kinetin concentration is preferably 0.8 to 1.2 mg/L, and most preferably 1.0 mg/L.

The silver nitrate concentration in the liquid decontamination medium is preferably at least 0.1 mg/L, more preferably at least 0.3 mg/L, still more preferably at least 0.5 mg/L. The silver nitrate concentration is preferably not more than 5.0 mg/L, more preferably not more than 3.0 mg/L.

The pH of the liquid decontamination medium is not particularly limited, but is preferably 4.0 to 10.0, more preferably 5.0 to 6.0. The decontamination temperature (the temperature of the liquid decontamination medium) is preferably 0° C. to 40° C., more preferably 20° C. to 36° C., still more preferably 22° C. to 30° C., particularly preferably 24° C. to 28° C.

Among the conditions indicated above, it is particularly preferred that the plant growth hormone is a cytokinin plant hormone, particularly benzyladenine, at a concentration of 4.0 to 6.0 mg/L, and the carbapenem antibiotic is present at a concentration of 10 to 500 mg/L (10 to 100 mg/L particularly in the case of meropenem).

In the decontamination step, after the shoot obtained in the infection step or the shoot obtained in the co-culture step is washed with the liquid decontamination medium, the shoot is separated from the liquid decontamination medium using, for example, filter paper, and the separated shoot is cultured in a decontamination medium.

The decontamination medium may be a liquid or a solid, but solid culture is preferred because a more effective decontamination can be achieved by plating and culture on the medium. When the decontamination medium is a liquid medium, static culture or shake culture may be carried out.

The decontamination medium may be as described for the liquid decontamination medium above. However, the carbapenem antibiotic concentration in the decontamination medium is preferably 10 to 500 mg/L. Due to the use of a carbapenem antibiotic as the decontaminating agent, the Agrobacterium can be thoroughly decontaminated by culture in the decontamination medium, even when the concentration of the decontaminating agent used for decontamination of the Agrobacterium is reduced to low levels as indicated above, and therefore the adverse effects on the plant arising from decontamination of the Agrobacterium can be minimized to prevent a decline in the survival rate of the transformed tissue fragment. The carbapenem antibiotic concentration is more preferably at least 12.5 mg/L, still more preferably at least 20 mg/L, further preferably at least 30 mg/L. It is also more preferably not more than 250 mg/L, still more preferably not more than 125 mg/L, further preferably not more than 100 mg/L, particularly preferably not more than 70 mg/L. A thorough decontamination of the Agrobacterium may not be achieved at less than 10 mg/L. Also, at above 500 mg/L the carbapenem antibiotic concentration is too high and can cause adverse effects not only on the Agrobacterium but even on shoot survival.

When the decontamination medium is used as a solid medium, the medium may be converted into a solid using a solidifying agent. There are no particular limitations on the solidifying agent, and examples include agar, gellan gum (e.g. Gelrite, Phytagel), agarose, and so forth.

When the decontamination medium is a solid medium, the solidifying agent concentration in the decontamination medium is preferably at least 0.1 mass %, more preferably at least 0.2 mass %, still more preferably at least 0.5 mass %. The solidifying agent concentration is preferably not more than 2.0 mass %, more preferably not more than 1.1 mass %, still more preferably not more than 0.8 mass %.

Culture in the decontamination medium is usually carried out in a controlled environment in which culture conditions such as temperature, light cycle, and so forth are managed. The culture conditions may be selected as appropriate, but, for example, the culture temperature is preferably 0° C. to 40° C., more preferably 20° C. to 40° C., still more preferably 25° C. to 35° C. Culture may be carried out in the dark or in the light, and the light conditions may be, for example, under a 14-16 h light cycle at 12.5 μmol/m²/s. There are no particular limitations on the culture time, but culture for 1 to 10 weeks is preferred, and culture for 3 to 5 weeks is more preferred. In addition, preferably subculture is performed while rewashing with the liquid decontamination medium every 1 to 4 weeks.

Among the conditions indicated above, it is particularly preferred that the plant growth hormone is a cytokinin plant hormone, particularly benzyladenine, at a concentration of 4.0 to 6.0 mg/L, the carbapenem antibiotic is present at a concentration of 10 to 500 mg/L (10 to 100 mg/L particularly in the case of meropenem), and the culture temperature is 25° C. to 35° C.

### (Selective Culture Step)

The selective culture step can be carried out by procedures commonly used in the Agrobacterium method. The transformed shoot can be sorted out from the untransformed shoot through this step.

The selective culture step includes culturing the shoot decontaminated in the decontamination step in a selective culture medium. The culture conditions for the selective culture step are not particularly limited as long as the conditions allow the transformed shoot (the shoot that has acquired the target gene) to be selectively grown.

The selective culture medium may be a liquid or a solid. When the selective culture medium is a liquid medium, static culture or shake culture may be carried out.

The selective culture medium may be prepared by adding a selective reagent corresponding to the selective marker gene to any base medium such as any of the basal media as described above, and modified basal media obtained by altering the composition of the basal media. Preferred thereamong are those prepared by adding the selective reagent to MS medium, B5 medium, or WP medium, more preferably to MS medium. As necessary, a plant growth hormone and a carbon source may be added. The plant growth hormones and carbon sources as described for the induction medium can be suitably used here. Moreover, the selective culture medium preferably further contains silver nitrate as described for the induction medium. In addition, active carbon is preferably also incorporated in order to prevent growth inhibitors from accumulating on the shoot.

There are no particular limitations on the selective reagent corresponding to the selective marker gene, and those skilled in the art can make an appropriate selection according to the selective marker gene used. By culturing the shoot, i.e. the shoot decontaminated in the decontamination step (a mixture of transformed and untransformed shoots), the transformed shoot can then grow in the medium owing to the selective reagent-resistance gene introduced along with the target gene, while the untransformed shoot does not grow in the medium. Thus, the transformed shoot can be selectively grown by culturing the mixture of transformed and untransformed shoots in a medium supplemented with a selective reagent corresponding to the selective marker gene. Accordingly, in another suitable embodiment of the present invention, the production method of the present invention further includes a selective culture step of culturing the cultured tissue fragment decontaminated in the decontamination step in a selective culture medium containing the selective reagent to select the tissue fragment transformed in the transformation step.

Paromomycin is particularly preferred as the selective reagent because then the selective growth of the transformed shoot of the target rubber-producing plant can occur with higher probability.

The concentration of the selective reagent in the selective culture medium is preferably 25 to 300 mg/L. It is more preferably at least 30 mg/L, still more preferably at least 40 mg/L, particularly preferably at least 50 mg/L. It is also more preferably not more than 290 mg/L, still more preferably not more than 280 mg/L. At less than 25 mg/L, the growth of the untransformed shoot cannot be sufficiently inhibited and the transformed shoot cannot be selectively grown. Also, the addition of the selective reagent at above 300 mg/L may have no substantial effect on the selectivity for the transformed shoot, which is uneconomical.

The suitable composition of the selective culture medium varies depending on the type of plant, but the composition is usually as follows, particularly in the case of Hevea brasiliensis.

The carbon source concentration in the selective culture medium is preferably at least 0.1 mass %, more preferably at least 1 mass %, still more preferably at least 2 mass %, particularly preferably at least 3 mass %. The carbon source concentration is preferably not more than 10 mass %, more preferably not more than 6 mass %, still more preferably not more than 5 mass %, further preferably not more than 4 mass %.

Preferably, substantially no auxin plant hormone is added to the selective culture medium, and the auxin plant hormone concentration in the selective culture medium is in particular preferably not more than 1.0 mg/L, more preferably not more than 0.1 mg/L, still more preferably not more than 0.05 mg/L, particularly preferably not more than 0.01 mg/L.

When a cytokinin plant hormone is added to the selective culture medium, the cytokinin plant hormone concentration in the selective culture medium is preferably at least 0.01 mg/L, more preferably at least 0.1 mg/L, still more preferably at least 0.5 mg/L, further preferably at least 0.8 mg/L, particularly preferably at least 1.0 mg/L, most preferably at least 3.0 mg/L. The cytokinin plant hormone concentration is preferably not more than 10.0 mg/L, more preferably not more than 7.0 mg/L, still more preferably not more than 6.0 mg/L.

Particularly when benzyladenine is used as the cytokinin plant hormone, the benzyladenine concentration is preferably 4.0 to 6.0 mg/L, and most preferably 5.0 mg/L. When, on the other hand, kinetin is used as the cytokinin plant hormone, the kinetin concentration is preferably 0.8 to 1.2 mg/L, and most preferably 1.0 mg/L.

The silver nitrate concentration in the selective culture medium is preferably at least 0.1 mg/L, more preferably at least 0.3 mg/L, still more preferably at least 0.5 mg/L. The silver nitrate concentration is preferably not more than 5.0 mg/L, more preferably not more than 3.0 mg/L.

The active carbon concentration in the selective culture medium is preferably at least 0.01 mass %, more preferably at least 0.03 mass %. The active carbon concentration is preferably not more than 1.0 mass %, more preferably not more than 0.1 mass %.

When the selective culture medium is used as a solid medium, the medium may be converted into a solid using a solidifying agent as described for the induction medium. There are no particular limitations on the solidifying agent, and examples include agar, gellan gum (e.g. Gelrite, Phytagel), agarose, and so forth.

When the selective culture medium is a solid medium, the solidifying agent concentration in the selective culture medium is preferably at least 0.1 mass %, more preferably at least 0.2 mass %, still more preferably at least 0.5 mass %. The solidifying agent concentration is preferably not more than 2.0 mass %, more preferably not more than 1.1 mass %, still more preferably not more than 0.8 mass %.

The pH of the selective culture medium is not particularly limited, but is preferably 5.0 to 7.0, more preferably 5.6 to 6.5.

Culture in the selective culture medium is usually carried out in a controlled environment in which culture conditions such as temperature, light cycle, and so forth are managed. The culture conditions may be selected as appropriate, but, for example, the culture temperature is preferably 0° C. to 40° C., more preferably 20° C. to 40° C., still more preferably 25° C. to 35° C. Culture may be carried out in the dark or in the light, and the light conditions may be, for example, under a 14-16 h light cycle at 12.5 μmol/m²/s. There are no particular limitations on the culture time as long as the effects of the present invention are achieved, but culture for 2 to 20 weeks is preferred, culture for 4 to 18 weeks is more preferred, culture for 6 to 15 weeks is still more preferred, and culture for 8 to 12 weeks is particularly preferred. In addition, preferably subculture is performed every 1 to 4 weeks.

The selective culture step may be carried out once or may be repeated a plurality of times. When the selective culture step is repeated a plurality of times, the concentration of the selective reagent in the selective culture medium may be the same each time or may be varied at least once. In particular, in another suitable embodiment, the selective culture step is repeated a plurality of times during which the concentration of the selective reagent in the selective culture medium is increased in stages because this allows the transformed shoot to be more selectively selected.

Among the conditions indicated above, it is particularly preferred that the plant growth hormone is a cytokinin plant hormone, particularly benzyladenine, at a concentration of 4.0 to 6.0 mg/L, the selective reagent (paromomycin) is present at a concentration of 25 to 300 mg/L, and the culture temperature is 25 to 35° C. Further, it is most preferred that the selective culture step is repeated a plurality of times by subculture, during which the concentration of the selective reagent (paromomycin) in the selective culture medium is increased in stages; specifically, culture is carried out at a selective reagent concentration of 30 to 70 mg/L for 7 to 21 days, more preferably 10 to 18 days, still more preferably 12 to 16 days, then at a selective reagent concentration of 75 to 120 mg/L for 7 to 21 days, more preferably 10 to 18 days, still more preferably 12 to 16 days, then at a selective reagent concentration of 125 to 170 mg/L for 7 to 21 days, more preferably 10 to 18 days, still more preferably 12 to 16 days, then at a selective reagent concentration of 175 to 220 mg/L for 7 to 21 days, more preferably 10 to 18 days, still more preferably 12 to 16 days, and then at a selective reagent concentration of 225 to 280 mg/L for 7 to 21 days, more preferably 10 to 18 days, still more preferably 12 to 16 days.

As described above, according to the selective culture step, by culturing in the selective culture medium the shoot obtained in the infection step or the co-culture step and then decontaminated in the decontamination step (a mixture of transformed and untransformed shoots), the transformed shoot can be selectively grown and thus can be sorted out from the untransformed shoot. The transformed shoot selected in this selective culture step is subjected to a subsequent regeneration step.

The transformed shoot transfected with the target gene or the like can also be mass propagated by subjecting the transformed shoot selected as above to the previously described propagation step.

Whether the thus selected transformed shoots have actually been transformed can be determined by conventional methods, such as by DNA extraction from the shoots followed by PCR analysis of whether the target gene or the like has been introduced, or by incorporation of a reporter gene, e.g. GUS gene or GFP gene, into the gene construct followed by GUS or GFP observation.

When, as described above, a genetically modified transgenic plant is produced by a production method including a culture step of culturing a tissue fragment derived from a rubber-producing plant to obtain a cultured tissue fragment, a transformation step of transforming, with a gene construct, the cultured tissue fragment obtained in the culture step, and a regeneration step of regenerating a plant from the tissue fragment transformed in the transformation step, wherein the transformation step includes an infection step of culturing the cultured tissue fragment in the presence of an Agrobacterium that has been transformed with a gene construct, and a decontamination step of culturing the cultured tissue fragment obtained in the infection step in a decontamination medium containing a carbapenem antibiotic to decontaminate the Agrobacterium, due to the use of the carbapenem antibiotic for decontamination of the Agrobacterium, the concentration of the decontaminating agent used to decontaminate the Agrobacterium can be reduced to low levels, and the Agrobacterium can be thoroughly decontaminated in a short period of time. Therefore, the adverse effects on the plant arising from decontamination of the Agrobacterium can be minimized to prevent a decline in the survival rate of the transformed tissue fragment. Moreover, by incorporating in the gene construct a paromomycin-resistance gene that confers resistance to paromomycin followed by a selective culture step of culturing the cultured tissue fragment decontaminated in the decontamination step in a paromomycin-containing selective culture medium to select the tissue fragment transformed in the transformation step, the selective growth of the transformed shoot of the target rubber-producing plant can occur with higher probability. Accordingly, the method can thereby particularly increase the efficiency of producing the genetically modified transgenic plant. Thus, a genetically modified transgenic rubber-producing plant can be more efficiently produced in a short period of time.

### (Regeneration Step)

The regeneration step includes regenerating a plant from the tissue fragment transformed in the transformation step, and more preferably from the transformed tissue fragment selected in the selective culture step.

There are no particular limitations on the regeneration method as long as it can regenerate a plant from the transformed tissue fragment, but, for example, plant regeneration may be carried out by subjecting the transformed tissue fragment to a rooting step that includes culturing the transformed tissue fragment in a rooting induction medium to induce rooting. Further, the tissue fragment rooted in the rooting step can be grown larger by transplanting the rooted tissue fragment to a cultivation soil for acclimatization. Thus, in another suitable embodiment of the present invention, the regeneration step includes a rooting step of culturing the transformed tissue fragment in a rooting induction medium to induce rooting.

The rooting step is described in the following.

### (Rooting Step)

The rooting step includes culturing the transformed tissue fragment in a rooting induction medium to induce rooting. The transformed tissue fragment may be the tissue fragment (shoot) transformed in the transformation step, preferably the transformed tissue fragment (shoot) selected in the selective culture step.

The rooting induction medium may be a liquid or a solid, but solid culture is preferred because rooting is facilitated by culture of the shoot inserted in the medium. When the rooting induction medium is a liquid medium, static culture or shake culture may be carried out.

The rooting induction medium contains a plant growth hormone and a carbon source, and examples of the plant growth hormone include auxin plant hormones and/or cytokinin plant hormones. Among these, auxin plant hormones are preferred.

The auxin plant hormones as described for the induction medium may be used as the auxin plant hormone here. Among them, 2,4-dichlorophenoxyacetic acid, 1-naphthaleneacetic acid, indole-3-butyric acid, and indole-3-acetic acid are preferred, with indole-3-butyric acid being more preferred.

The cytokinin plant hormones as described for the induction medium may be used as the cytokinin plant hormone here. Among them, benzyladenine, kinetin, and zeatin are preferred, with benzyladenine or kinetin being more preferred.

There are no particular limitations on the carbon source used in the rooting induction medium, and the carbon sources as described for the induction medium may be used here, among which sucrose is preferred.

The rooting induction medium preferably further contains silver nitrate as described for the induction medium.

The rooting induction medium may be a base medium such as any of the basal media as described for the induction medium, and modified basal media obtained by altering the composition of the basal media. MS medium, B5 medium, and WP medium, among others, are preferred, and MS medium and modified MS media obtained by altering the composition of MS medium are more preferred.

When the rooting induction medium is used as a solid medium, the medium may be converted into a solid using a solidifying agent. There are no particular limitations on the solidifying agent, and examples include agar, gellan gum (e.g. Gelrite, Phytagel), agarose, and so forth.

The suitable composition and culture conditions of the rooting induction medium vary depending on the type of plant and also vary depending on whether the medium is a liquid medium or a solid medium, but the composition is usually as follows, particularly in the case of Hevea brasiliensis.

The carbon source concentration in the rooting induction medium is preferably at least 0.1 mass %, more preferably at least 1.0 mass %. The carbon source concentration is preferably not more than 10 mass %, more preferably not more than 5.0 mass %.

When an auxin plant hormone is added to the rooting induction medium, the auxin plant hormone concentration in the rooting induction medium is preferably at least 0.5 mg/L, more preferably at least 1.0 mg/L, still more preferably at least 3.0 mg/L. The auxin plant hormone concentration is preferably not more than 10 mg/L, more preferably not more than 6.0 mg/L, still more preferably not more than 5.0 mg/L.

Preferably, substantially no cytokinin plant hormone is added to the rooting induction medium, and the concentration is in particular preferably not more than 1.0 mg/L, more preferably not more than 0.1 mg/L, still more preferably not more than 0.05 mg/L, particularly preferably not more than 0.01 mg/L.

The silver nitrate concentration in the rooting induction medium is preferably at least 0.1 mg/L, more preferably at least 0.3 mg/L, still more preferably at least 0.5 mg/L. The silver nitrate concentration is preferably not more than 5.0 mg/L, more preferably not more than 3.0 mg/L.

The pH of the rooting induction medium is preferably 4.0 to 10.0, more preferably 5.0 to 6.5, still more preferably 5.5 to 6.0.

The rooting step is usually carried out in a controlled environment in which culture conditions such as temperature, light cycle, and so forth are managed. The culture conditions may be selected as appropriate, but, for example, the culture temperature is preferably 0° C. to 40° C., more preferably 20° C. to 40° C., still more preferably 25° C. to 35° C. Culture may be carried out in the dark or in the light, and the light conditions may be, for example, under a 14-16 h light cycle at 12.5 μmol/m²/s. There are no particular limitations on the culture time, but culture for 1 to 10 weeks is preferred, and culture for 4 to 8 weeks is more preferred.

When the rooting induction medium is a solid medium, the solidifying agent concentration in the rooting induction medium is preferably at least 0.1 mass %, more preferably at least 0.2 mass %, still more preferably at least 0.5 mass %. The solidifying agent concentration is preferably not more than 2.0 mass %, more preferably not more than 1.1 mass %, still more preferably not more than 0.8 mass %.

Among the conditions indicated above, it is particularly preferred that the plant growth hormone is an auxin plant hormone, particularly indole-3-butyric acid, at a concentration of 3.0 to 6.0 mg/L, and the culture temperature is 25° C. to 35° C.

As described above, by culturing the transformed shoot in the rooting induction medium, rooting can be induced to obtain a rooted shoot and therefore a clone plantlet which is a complete plant can be formed.

In order to mass propagate an improved clone plantlet, the clone plantlet formed as above can also be repeatedly subjected to the previously described propagation step.

As has been described above, the production method of the present invention includes a culture step of culturing a tissue fragment derived from a rubber-producing plant to obtain a cultured tissue fragment, a transformation step of transforming, with a gene construct, the cultured tissue fragment obtained in the culture step, and a regeneration step of regenerating a plant from the tissue fragment transformed in the transformation step, wherein the transformation step includes an infection step of culturing the cultured tissue fragment in the presence of an Agrobacterium that has been transformed with a gene construct, and a decontamination step of culturing the cultured tissue fragment obtained in the infection step in a decontamination medium containing a carbapenem antibiotic to decontaminate the Agrobacterium. Such a method allows a genetically modified transgenic plant to be efficiently produced in a short period of time. Thus, another aspect of the present invention is a genetically modified transgenic plant produced by the transgenic plant production method for producing a genetically modified plant of the present invention.

In summary, the sequence of a series of steps is described for an example of the transgenic plant production method for producing a genetically modified plant of the present invention, in which Hevea brasiliensis is used as the rubber-producing plant. First, a tissue containing an axillary bud is collected from a mature tree of Hevea brasiliensis and cultured in an induction medium to form a shoot (induction step); the formed shoot is cultured in an elongation medium (elongation step); the elongated shoot is collected and subdivided and the subdivided shoot is cultured in an induction medium to form a shoot (propagation step); the formed shoot is cultured in the presence of an Agrobacterium that has been transformed with a gene construct, to infect the shoot with the Agrobacterium (infection step); the shoot obtained in the infection step is cultured in a co-culture medium (co-culture step); the shoot obtained in the co-culture step is cultured in a decontamination medium containing a carbapenem antibiotic to decontaminate the Agrobacterium (decontamination step); the shoot decontaminated in the decontamination step is cultured in a selective culture medium to selectively grow the transformed shoot (selective culture step); and the transformed shoot is cultured in a rooting induction medium to induce rooting and growth (rooting step).

As described above, the transgenic plant production method for producing a genetically modified plant of the present invention can efficiently produce a genetically modified transgenic plant (transformed plant) in a short period of time. Such a method in which Hevea brasiliensis is used as the target plant can expand the possibilities of natural rubber derived from Hevea brasiliensis in industrial applications of isoprenoids. In addition, the method can contribute to the mass culture and molecular breeding of isoprenoid-producing plants such as Hevea brasiliensis.

### EXAMPLES

The present invention is specifically described with reference to examples, but the present invention is not limited only to these.

The chemicals used in the examples are collectively described in the following.
BA: benzyladenine
silver nitrate: silver nitrate from Merck
agar: agar (powder) from FLUKA
acetosyringone: acetosyringone from Tokyo Chemical Industry Co., Ltd.
meropenem: meropenem (Meropen for drip infusion vial) from Sumitomo Dainippon Pharma Co., Ltd.
paromomycin: paromomycin (paromomycin sulfate) from Wako Pure Chemical Industries, Ltd.
hygromycin: hygromycin (hygromycin B) from Wako Pure Chemical Industries, Ltd.
cefotaxime: cefotaxime (product name: CEFOX) from UTOPIAN
Agrobacterium: strain EHA105
Hevea brasiliensis: Hevea brasiliensis sourced from the

Arboricultural Research Institute of the University of Tokyo

### Example 1

### <Induction Step>

Tissues containing a node, axillary bud, or apical bud were collected from mature trees and saplings of Hevea brasiliensis. In addition, tissues containing a node, axillary bud, or apical bud were collected from seedlings obtained by in vitro aseptic germination and culture of Hevea brasiliensis seeds (aseptic seedlings).

Next, the tissues containing a node, axillary bud, or apical bud collected from the mature trees and saplings were washed with running water and then with 70 mass % ethanol, subsequently sterilized with an aqueous sodium hypochlorite solution diluted at approximately 5 to 10 volume %, and washed with sterile water.

Next, the sterilized tissues and the tissues derived from the aseptic seedlings were collectively inserted into an induction medium (solid medium) and cultured (induction step). The induction medium was prepared by adding benzyladenine (BA), silver nitrate, active carbon, and sucrose at 5.0 mg/L, 1.0 mg/L, 0.05 mass %, and 3.0 mass %, respectively, to MS medium (disclosed on pp. 20-36 of Shokubutsu Saibo Kogaku Nyumon (Introduction to Plant Cell Engineering), Japan Scientific Societies Press), adjusting the pH of the medium to 5.7, and adding thereto 0.75 mass % of agar followed by sterilization in an autoclave (121° C., 20 minutes) and cooling in a clean bench.

The Hevea brasiliensis tissues were inserted into the induction medium (solid medium) and cultured for 4 weeks under a 16 h light cycle at 12.5 μmol/m²/s at a culture temperature of 28° C. to cause shoot induction. A good shoot induction was achieved and the formation of shoots and multiple shoots was observed. The formed shoots and multiple shoots were subcultured by transfer every 4 weeks to an induction medium having the same composition.

Then, the shoots that had grown to about 3 cm by subculture were divided to 1-2.5 cm portions with the node, axillary bud, or apical bud left therein to prepare a source material for transformation.

### <Agrobacterium Preparation Step>

An Agrobacterium (Agrobacterium tumefaciens, EHA105) transfected with a binary vector pCAMBIA2300 (trade name, Marker Gene Technologies, Inc) into which a paromomycin-resistance gene (selective marker gene) had been inserted along with GFP (green fluorescent protein) gene and GUS (β-glucuronidase) genes was shake cultured in YEB medium for 24 hours at a culture temperature of 28° C. Culture was continued until the absorbance measured at 600 nm (OD 600) reached approximately 1.0, and the bacteria were then harvested by centrifugal separation followed by adjustment to OD600=0.6 using a suspending solution (MS liquid medium supplemented with 5.0 mg/L BA, 3.0 mass % sucrose, and 1.0 mg/L silver nitrate).

### <Infection Step and Co-Culture Step>

25 mL of the prepared Agrobacterium suspension and five source material shoots for transformation prepared in the induction step were introduced into a 50-mL tube and gently shaken for 30 minutes at 28° C. (infection step). After shaking, the shoots were placed on sterilized filter paper to thoroughly remove the excess suspension. These shoots were inserted into a co-culture medium (solid medium) and co-cultured for 3 days in the dark (at an illuminance of less than 0.1 lx) at a culture temperature of 28° C. (co-culture step).

The co-culture medium was prepared by adding benzyladenine (BA), silver nitrate, sucrose, and acetosyringone at 5.0 mg/L, 1.0 mg/L, 3.0 mass %, and 200 μM, respectively, to MS medium, adjusting the pH of the medium to 5.7, and adding thereto 0.75 mass % of agar followed by sterilization in an autoclave (121° C., 20 minutes) and cooling in a clean bench.

### <Decontamination Step>

The co-cultured shoots were taken out and washed by immersion for 10 minutes in a liquid decontamination medium (MS liquid medium supplemented with 5.0 mg/L BA, 3.0 mass % sucrose, 1.0 mg/L silver nitrate, and 50 mg/L meropenem).

After washing, the shoots were placed on sterilized filter paper to wipe off the excess moisture. The shoots were inserted into a decontamination medium (solid medium) and cultured for 4 weeks under a 14 h light cycle at 12.5 μmol/m²/s at a culture temperature of 28° C. (decontamination step). Here, the shoots were subcultured by transfer every other week to a decontamination medium having the same composition. As a result, no Agrobacterium colony was found by visual observation.

The decontamination medium was prepared by adding benzyladenine (BA), silver nitrate, sucrose, and meropenem at 5.0 mg/L, 1.0 mg/L, 3.0 mass %, and 50 mg/L, respectively, to MS medium, adjusting the pH of the medium to 5.8, and adding thereto 0.75 mass % of agar followed by sterilization in an autoclave (121° C., 20 minutes) and cooling in a clean bench.

The shoot survival rate after culture in the decontamination medium was calculated using the following formula.

(Shoot survival rate (%))={(the number of shoots for which survival without exhaustion was observed)/(the number of shoots subjected to the decontamination step)}×100

The shoot survival rate in Example 1 was 100%.

### <Selective Culture Step>

The shoots cultured in the decontamination medium were taken out and these shoots were inserted into a selective culture medium (solid medium) and cultured for 10 weeks under a 14 h light cycle at 12.5 mol/m²/s at a culture temperature of 28° C. in order to select the transgenic individuals (selective culture step). Here, the shoots were subcultured by transfer every two weeks to selective culture media having the following compositions.

(Culture from Week 0 to Week 2)

The selective culture medium used was prepared by adding benzyladenine (BA), silver nitrate, active carbon, sucrose, and paromomycin at 5.0 mg/L, 1.0 mg/L, 0.05 mass %, 3.0 mass %, and 50 mg/L, respectively, to MS medium, adjusting the pH of the medium to 5.7, and adding thereto 0.75 mass % of agar followed by sterilization in an autoclave (121° C., 20 minutes) and cooling in a clean bench.

(Culture from Week 2 to Week 4)

The selective culture medium used was prepared by adding benzyladenine (BA), silver nitrate, active carbon, sucrose, and paromomycin at 5.0 mg/L, 1.0 mg/L, 0.05 mass %, 3.0 mass %, and 100 mg/L, respectively, to MS medium, adjusting the pH of the medium to 5.7, and adding thereto 0.75 mass % of agar followed by sterilization in an autoclave (121° C., 20 minutes) and cooling in a clean bench.

(Culture from Week 4 to Week 6)

The selective culture medium used was prepared by adding benzyladenine (BA), silver nitrate, active carbon, sucrose, and paromomycin at 5.0 mg/L, 1.0 mg/L, 0.05 mass %, 3.0 mass %, and 150 mg/L, respectively, to MS medium, adjusting the pH of the medium to 5.7, and adding thereto 0.75 mass % of agar followed by sterilization in an autoclave (121° C., 20 minutes) and cooling in a clean bench.

(Culture from Week 6 to Week 8)

The selective culture medium used was prepared by adding benzyladenine (BA), silver nitrate, active carbon, sucrose, and paromomycin at 5.0 mg/L, 1.0 mg/L, 0.05 mass %, 3.0 mass %, and 200 mg/L, respectively, to MS medium, adjusting the pH of the medium to 5.7, and adding thereto 0.75 mass % of agar followed by sterilization in an autoclave (121° C., 20 minutes) and cooling in a clean bench.

(Culture from Week 8 to Week 10)

The selective culture medium used was prepared by adding benzyladenine (BA), silver nitrate, active carbon, sucrose, and paromomycin at 5.0 mg/L, 1.0 mg/L, 0.05 mass %, 3.0 mass %, and 250 mg/L, respectively, to MS medium, adjusting the pH of the medium to 5.7, and adding thereto 0.75 mass % of agar followed by sterilization in an autoclave (121° C., 20 minutes) and cooling in a clean bench.

The shoot development ratio and shoot selection efficiency (transformant acquisition efficiency) after culture in the selective culture media were calculated using the following formulas.

(Shoot development ratio (%))={(the number of shoots for which new shoot development was observed)/(the number of shoots subjected to the selective culture step)}×100

(Shoot selection efficiency (%))={(the number of shoots for which gene transfer was observed)/(the number of shoots for which the outcome of gene transfer was determined (the number of shoots subjected to GFP observation))}×100

The shoot selection efficiency is an indicator of the proportion of sampled test individuals for which gene transfer was observed when a sampling test was carried out in which GFP observation was performed on a number of individuals sampled from among the observed new shoots developed after the selective culture step.

The shoot development ratio in Example 1 was 90%. In addition, the shoot selection efficiency based on the sampling test was 100%. Thus, shoot development and growth were observed after culture in the selective culture media, and the transgenic individuals could be selected. The selected transgenic individuals were further cultured by continuing subculture by transfer to a medium having the same composition as the selective culture medium used for the culture from week 8 to week 10 in the above selective culture step to obtain shoots and multiple shoots.

Thus, genetically modified transgenic plants could be efficiently produced in a short period of time by a production method including a culture step of culturing a tissue fragment derived from a rubber-producing plant to obtain a cultured tissue fragment, a transformation step of transforming, with a gene construct, the cultured tissue fragment obtained in the culture step, and a regeneration step of regenerating a plant from the tissue fragment transformed in the transformation step, wherein the transformation step includes an infection step of culturing the cultured tissue fragment in the presence of an Agrobacterium that has been transformed with a gene construct, and a decontamination step of culturing the cultured tissue fragment obtained in the infection step in a decontamination medium containing a carbapenem antibiotic to decontaminate the Agrobacterium.

### Example 2

### <Induction Step>

A source material for transformation was prepared as in Example 1.

### <Agrobacterium Preparation Step>

An Agrobacterium (Agrobacterium tumefaciens, EHA105) transfected with a binary vector pCAMBIA2300 (trade name, Marker Gene Technologies, Inc) into which a paromomycin-resistance gene (selective marker gene) had been inserted along with GFP (green fluorescent protein), GUS (β-glucuronidase), and CPT (cis-prenyltransferase) genes was shake cultured in YEB medium for 24 hours at a culture temperature of 28° C. Culture was continued until the absorbance measured at 600 nm (OD 600) reached approximately 1.0, and the bacteria were then harvested by centrifugal separation followed by adjustment to OD600=0.6 using a suspending solution (MS liquid medium supplemented with 5.0 mg/L BA, 3.0 mass % sucrose, and 1.0 mg/L silver nitrate).

### <Infection Step and Co-Culture Step>

Infection and co-culture steps were performed as in Example 1, except that the Agrobacterium suspension prepared above was used.

### <Decontamination Step>

A decontamination step was performed as in Example 1. As a result, no Agrobacterium colony was found by visual observation.

The shoot survival rate after culture in the decontamination medium was calculated as in Example 1, and the shoot survival rate in Example 2 was found to be 100%.

### <Selective Culture Step>

The shoots cultured in the decontamination medium were taken out and subjected to a selective culture step as in Example 1.

The shoot development ratio and shoot selection efficiency (transformant acquisition efficiency) after culture in the selective culture media were calculated as in Example 1, and the shoot development ratio in Example 2 was found to be 60%; in addition, the shoot selection efficiency based on the sampling test was 80%. Thus, shoot development and growth were observed after culture in the selective culture media, and the transgenic individuals could be selected. The selected transgenic individuals were further cultured by continuing subculture by transfer to a medium having the same composition as the selective culture medium used for the culture from week 8 to week 10 in the above selective culture step to obtain shoots and multiple shoots.

Thus, genetically modified transgenic plants could be efficiently produced in a short period of time by a production method including a culture step of culturing a tissue fragment derived from a rubber-producing plant to obtain a cultured tissue fragment, a transformation step of transforming, with a gene construct, the cultured tissue fragment obtained in the culture step, and a regeneration step of regenerating a plant from the tissue fragment transformed in the transformation step, wherein the transformation step includes an infection step of culturing the cultured tissue fragment in the presence of an Agrobacterium that has been transformed with a gene construct, and a decontamination step of culturing the cultured tissue fragment obtained in the infection step in a decontamination medium containing a carbapenem antibiotic to decontaminate the Agrobacterium.

### Example 3

### <Induction Step>

A source material for transformation was prepared as in Example 1.

### <Agrobacterium Preparation Step>

An Agrobacterium (Agrobacterium tumefaciens, EHA105) transfected with a binary vector pCAMBIA1304 (trade name, Marker Gene Technologies, Inc) into which a hygromycin-resistance gene (selective marker gene) had been inserted along with GFP (green fluorescent protein) and GUS (β-glucuronidase) genes was shake cultured in YEB medium for 24 hours at a culture temperature of 28° C. Culture was continued until the absorbance measured at 600 nm (OD 600) reached approximately 1.0, and the bacteria were then harvested by centrifugal separation followed by adjustment to OD600=0.6 using a suspending solution (MS liquid medium supplemented with 5.0 mg/L BA, 3.0 mass % sucrose, and 1.0 mg/L silver nitrate).

### <Infection Step and Co-Culture Step>

Infection and co-culture steps were performed as in Example 1, except that the Agrobacterium suspension prepared above was used.

### <Decontamination Step>

A decontamination step was performed as in Example 1. As a result, no Agrobacterium colony was found by visual observation.

The shoot survival rate after culture in the decontamination medium was calculated as in Example 1, and the shoot survival rate in Example 3 was found to be 100%.

### <Selective Culture Step>

The shoots cultured in the decontamination medium were taken out and these shoots were inserted into a selective culture medium (solid medium) and cultured for 9 weeks under a 14 h light cycle at 12.5 μmol/m²/s at a culture temperature of 28° C. in order to select the transgenic individuals (selective culture step). Here, the shoots were subcultured by transfer every other week to a selective culture medium having the same composition.

The selective culture medium was prepared by adding benzyladenine (BA), silver nitrate, active carbon, sucrose, and hygromycin at 5.0 mg/L, 1.0 mg/L, 0.05 mass %, 3.0 mass %, and 5 mg/L, respectively, to MS medium, adjusting the pH of the medium to 5.7, and adding thereto 0.75 mass % of agar followed by sterilization in an autoclave (121° C., 20 minutes) and cooling in a clean bench.

### Comparative Example 1

### <Induction Step>

A source material for transformation was prepared as in Example 1.

### <Agrobacterium Preparation Step>

An Agrobacterium (Agrobacterium tumefaciens, EHA105) transfected with a binary vector pCAMBIA1304 (trade name, Marker Gene Technologies, Inc) into which a hygromycin-resistance gene (selective marker gene) had been inserted along with GFP (green fluorescent protein), GUS (β-glucuronidase), and CPT (cis-prenyltransferase) genes was shake cultured in YEB medium for 24 hours at a culture temperature of 28° C. Culture was continued until the absorbance measured at 600 nm (OD 600) reached approximately 1.0, and the bacteria were then harvested by centrifugal separation followed by adjustment to OD600=0.6 using a suspending solution (MS liquid medium supplemented with 5.0 mg/L BA, 3.0 mass % sucrose, and 1.0 mg/L silver nitrate).

### <Infection Step and Co-Culture Step>

Infection and co-culture steps were performed as in Example 1, except that the Agrobacterium suspension prepared above was used.

### <Decontamination Step>

The co-cultured shoots were taken out and washed by immersion for 10 minutes in a liquid decontamination medium (MS liquid medium supplemented with 5.0 mg/L BA, 3.0 mass % sucrose, 1.0 mg/L silver nitrate, and 1000 mg/L cefotaxime). This washing operation was repeated five times.

After washing, the shoots were placed on sterilized filter paper to wipe off the excess moisture. The shoots were inserted into a decontamination medium (solid medium) and cultured for 4 weeks under a 14 h light cycle at 12.5 μmol/m²/s at a culture temperature of 28° C. (decontamination step). Here, the shoots were subcultured by transfer every other week to a decontamination medium having the same composition. As a result, no Agrobacterium colony was found by visual observation.

The decontamination medium was prepared by adding benzyladenine (BA), silver nitrate, sucrose, and cefotaxime at 5.0 mg/L, 1.0 mg/L, 3.0 mass %, and 500 mg/L, respectively, to MS medium, adjusting the pH of the medium to 5.8, and adding thereto 0.75 mass % of agar followed by sterilization in an autoclave (121° C., 20 minutes) and cooling in a clean bench.

The shoot survival rate after culture in the decontamination medium was calculated as in Example 1, and the shoot survival rate in Comparative Example 1 was found to be 10%.

### <Selective Culture Step>

The shoots cultured in the decontamination medium were taken out and these shoots were inserted into a selective culture medium (solid medium) and cultured for 10 weeks under a 14 h light cycle at 12.5 μmol/m²/s at a culture temperature of 28° C. in order to select the transgenic individuals (selective culture step). Here, the shoots were subcultured by transfer every two weeks to a selective culture medium having the same composition.

The selective culture medium was prepared by adding benzyladenine (BA), silver nitrate, active carbon, sucrose, and hygromycin at 5.0 mg/L, 1.0 mg/L, 0.05 mass %, 3.0 mass %, and 5 mg/L, respectively, to MS medium, adjusting the pH of the medium to 5.7, and adding thereto 0.75 mass % of agar followed by sterilization in an autoclave (121° C., 20 minutes) and cooling in a clean bench.

The shoot development ratio after culture in the selective culture medium was calculated as in Example 1, and the shoot development ratio in Comparative Example 1 was found to be 0%.

### Comparative Example 2

### <Induction Step>

A source material for transformation was prepared as in Example 1.

### <Agrobacterium Preparation Step>

An agrobacterium suspension was prepared as in Comparative Example 1.

### <Infection Step and Co-Culture Step>

Infection and co-culture steps were performed as in Example 1, except that the Agrobacterium suspension prepared above was used.

### <Decontamination Step>

The co-cultured shoots were taken out and washed by immersion for 10 minutes in a liquid decontamination medium (MS liquid medium supplemented with 5.0 mg/L BA, 3.0 mass % sucrose, 1.0 mg/L silver nitrate, and 1000 mg/L cefotaxime). This washing operation was repeated eight times.

After washing, the shoots were placed on sterilized filter paper to wipe off the excess moisture. The shoots were inserted into a decontamination medium (solid medium) and cultured for 4 weeks under a 14 h light cycle at 12.5 μmol/m²/s at a culture temperature of 28° C. (decontamination step). Here, the shoots were subcultured by transfer every other week to a decontamination medium having the same composition. As a result, no Agrobacterium colony was found by visual observation.

The decontamination medium was prepared by adding benzyladenine (BA), silver nitrate, sucrose, and cefotaxime at 5.0 mg/L, 1.0 mg/L, 3.0 mass %, and 400 mg/L, respectively, to MS medium, adjusting the pH of the medium to 5.8, and adding thereto 0.75 mass % of agar followed by sterilization in an autoclave (121° C., 20 minutes) and cooling in a clean bench.

The shoot survival rate after culture in the decontamination medium was calculated as in Example 1, and the shoot survival rate in Comparative Example 2 was found to be 30%.

### <Selective Culture Step>

The shoots cultured in the decontamination medium were taken out and these shoots were inserted into a selective culture medium (solid medium) and cultured for 10 weeks under a 14 h light cycle at 12.5 μmol/m²/s at a culture temperature of 28° C. in order to select the transgenic individuals (selective culture step). Here, the shoots were subcultured by transfer every two weeks to a selective culture medium having the same composition.

The selective culture medium was prepared by adding benzyladenine (BA), silver nitrate, active carbon, sucrose, and hygromycin at 5.0 mg/L, 1.0 mg/L, 0.05 mass %, 3.0 mass %, and 50 mg/L, respectively, to MS medium, adjusting the pH of the medium to 5.7, and adding thereto 0.75 mass % of agar followed by sterilization in an autoclave (121° C., 20 minutes) and cooling in a clean bench.

The shoot development ratio after culture in the selective culture medium was calculated as in Example 1, and the shoot development ratio in Comparative Example 2 was found to be 0%.

### Comparative Example 3

### <Induction Step>

A source material for transformation was prepared as in Example 1.

### <Agrobacterium Preparation Step>

An agrobacterium suspension was prepared as in Comparative Example 1.

### <Infection Step and Co-Culture Step>

Infection and co-culture steps were performed as in Example 1, except that the Agrobacterium suspension prepared above was used.

### <Decontamination Step>

The co-cultured shoots were taken out and washed by immersion for 10 minutes in a liquid decontamination medium (MS liquid medium supplemented with 5.0 mg/L BA, 3.0 mass % sucrose, 1.0 mg/L silver nitrate, and 400 mg/L cefotaxime). This washing operation was repeated fourteen times.

After washing, the shoots were placed on sterilized filter paper to wipe off the excess moisture. The shoots were inserted into a decontamination medium (solid medium) and cultured for 4 weeks under a 14 h light cycle at 12.5 μmol/m²/s at a culture temperature of 28° C. (decontamination step). Here, the shoots were subcultured by transfer every other week to a decontamination medium having the same composition. As a result, no Agrobacterium colony was found by visual observation.

The decontamination medium was prepared by adding benzyladenine (BA), silver nitrate, sucrose, and cefotaxime at 5.0 mg/L, 1.0 mg/L, 3.0 mass %, and 200 mg/L, respectively, to MS medium, adjusting the pH of the medium to 5.8, and adding thereto 0.75 mass % of agar followed by sterilization in an autoclave (121° C., 20 minutes) and cooling in a clean bench.

The shoot survival rate after culture in the decontamination medium was calculated as in Example 1, and the shoot survival rate in Comparative Example 3 was found to be 20%.

### <Selective Culture Step>

The shoots cultured in the decontamination medium were taken out and these shoots were inserted into a selective culture medium (solid medium) and cultured for 10 weeks under a 14 h light cycle at 12.5 μmol/m²/s at a culture temperature of 28° C. in order to select the transgenic individuals (selective culture step). Here, the shoots were subcultured by transfer every two weeks to a selective culture medium having the same composition.

The selective culture medium was prepared by adding benzyladenine (BA), silver nitrate, active carbon, sucrose, and hygromycin at 5.0 mg/L, 1.0 mg/L, 0.05 mass %, 3.0 mass %, and 50 mg/L, respectively, to MS medium, adjusting the pH of the medium to 5.7, and adding thereto 0.75 mass % of agar followed by sterilization in an autoclave (121° C., 20 minutes) and cooling in a clean bench.

The shoot development ratio after culture in the selective culture medium was calculated as in Example 1, and the shoot development ratio in Comparative Example 3 was found to be 0%.

Thus, in Comparative Examples 1 to 3 using cefotaxime instead of the carbapenem antibiotic in the decontamination step for decontaminating the Agrobacterium, repeated washing with a high concentration of cefotaxime and culture in a medium supplemented with a high concentration of cefotaxime were required in order to decontaminate the Agrobacterium, and therefore the shoot survival rate was low and no shoot development was observed after culture in the selective culture medium.

1. A transgenic plant production method for producing a genetically modified plant, the production method comprising:
a culture step of culturing a tissue fragment derived from a rubber-producing plant to obtain a cultured tissue fragment;
a transformation step of transforming, with a gene construct, the cultured tissue fragment obtained in the culture step; and
a regeneration step of regenerating a plant from the tissue fragment transformed in the transformation step,
wherein the transformation step comprises:
an infection step of culturing the cultured tissue fragment in the presence of an Agrobacterium that has been transformed with a gene construct; and
a decontamination step of culturing the cultured tissue fragment obtained in the infection step in a decontamination medium containing a carbapenem antibiotic to decontaminate the Agrobacterium.

2. The transgenic plant production method according to claim 1, wherein the carbapenem antibiotic is meropenem.

3. The transgenic plant production method according to claim 1, wherein the carbapenem antibiotic is present in the decontamination medium at a concentration of 10 to 500 mg/L.

4. The transgenic plant production method according to claim 1, wherein
the gene construct contains a selective marker gene that confers resistance to a selective reagent, and
the production method further comprises a selective culture step of culturing the cultured tissue fragment decontaminated in the decontamination step in a selective culture medium containing the selective reagent to select the tissue fragment transformed in the transformation step.

5. The transgenic plant production method according to claim 4, wherein the selective reagent is paromomycin.

6. The transgenic plant production method according to claim 4, wherein the selective reagent is present in the selective culture medium at a concentration of 25 to 300 mg/L.

7. The transgenic plant production method according to claim 1, wherein the cultured tissue fragment is a shoot.

8. The transgenic plant production method according to claim 1, wherein the rubber-producing plant is a plant belonging to the genus Hevea.

9. The transgenic plant production method according to claim 1, wherein
the production method further comprises, after the culture step, a propagation step of collecting and subdividing the cultured tissue fragment obtained in the culture step, and culturing the subdivided cultured tissue fragment in an induction medium containing a plant growth hormone and a carbon source to obtain a cultured tissue fragment, and
the cultured tissue fragment obtained in the propagation step is subjected to the transformation step.

10. The transgenic plant production method according to claim 1, wherein the gene construct contains genetic material that is homologous to the genome of the rubber-producing plant.

11. The transgenic plant production method according to claim 1, wherein the gene construct contains genetic material that is heterologous to the genome of the rubber-producing plant.

12. The transgenic plant production method according to claim 1, wherein the regeneration step comprises a rooting step of culturing the transformed tissue fragment in a rooting induction medium to induce rooting.

13. A genetically modified transgenic plant, produced by the production method according to claim 1.
